(19) United States
(12) Patent Application Publication
(10) Pub. No.: US 2020/0340934 A1
(43) Pub. Date: Oct. 29, 2020

(54) INSTALLATION AND PROCESS OF FOLLOW-UP OF THE EVOLUTION OF THE QUALITY OF A LUBRICANT, METHOD OF FOLLOW-UP AND USE OF SUCH A METHOD FOR DETERMINING THE IRON CONTENT OF A LUBRICANT

(71) Applicant: TOTAL MARKETING SERVICES, Puteaux (FR)

(72) Inventors: Raphael JUSTON, Chambery (FR); Yann TROADEC, Grenoble (FR); François CHAUDOREILLE, Gresy sur Aix (FR); Jean-Philippe ROMAN, Septeme (FR); Arnaud AMIOT, Rueil Malmaison (FR)

(73) Assignee: Total Marketting Services, Puteaux (FR)

(21) Appl. No.: 16/306,137

(22) PCT Filed: Jun. 2, 2017
(86) PCT No.: PCT/EP2017/063427

§ 371 Date: Nov. 30, 2018

(30) Foreign Application Priority Data
Jun. 2, 2016 (FR) 1655022

(51) Int. Cl. G01N 23/223; B63B 79/30; G01N 33/28

(52) U.S. Cl. CPC G01N 23/223; B63B 79/30; G01N 33/2858; G01N 2223/652; G01N 2223/076; G01N 2223/507; G01N 33/2888

(57) ABSTRACT

An installation for following up the evolution of the quality of a lubricant circulating in equipment includes a conduit connected, upstream, to the piece of equipment and downstream to a recovery pan. A first controlled interruption valve circulates the lubricant in the conduit, a buffer reservoir accumulates lubricant, and a first bypass line is connected to the conduit, upstream from the first valve and to the buffer reservoir. A second controlled interruption valve circulates the lubricant in the first bypass line, a second discharge line discharges the lubricant, from the buffer reservoir to the recovery pan and a third controlled interruption valve circulates the lubricant in the second discharge line. A sensor determines content in a predetermined chemical element of a lubricant sample at the outlet of the buffer reservoir. This sensor includes an X-ray source and detector and a sample cell with a poly X-ray window.

The present invention relates to an installation for following up the evolution of the quality of a lubricant circulating in a piece of equipment, such as a ship engine. The invention also relates to a method for following up the evolution of the content of a predetermined chemical element of a lubricant, notably dissolved iron and/or particulate iron of a lubricant. Further, the invention relates to a method for following up the operation of a piece of equipment loaded onboard a ship.

The field of the invention is that of equipment for analyzing lubricants, notably for engines, in particular for marine engines.

In the field of internal combustion engines used on commercial ships, it is known that the situation of an engine should be followed up by analyzing the lubricant circulating in this engine. Such an analysis gives the possibility of detecting phenomena of wear and/or corrosion which tend to occur in an engine. In the past, the operation of engines was relatively stabilized and it was sufficient to control the quality of a lubricant on an on/off basis, during vessel hot-cold, in order to anticipate the maintenance operations to be carried out. Nowadays, engines are increasing elaborated and sensitive to phenomena of wear and/or of corrosion, so that analyses have to be carried out onboard ships and at sea, notably for tracking the global iron content of the lubricant, also called oil or engine oil, this content resulting from abrasion phenomena. The overall iron content of the lubricant comprises the iron content present in the lubricant in particulate form, for example as iron oxide or in its dissolved form, for example in an ionic form. This imposes training of personnel and of taking onboard an elaborate material, the operation of which is relatively difficult to control, even with the trained seagoing personnel. Further, this increases the work burden of the personnel.

WO-A-2010/046591 provides the use of an onboard system in which the oil leaving an engine is directed towards a functional component associated with a measurement system giving the possibility of determining its basicity index or its content of metal particles. In practice, the oil flow rate leaving the engine is low and the flow at the output of the engine consist of droplets which stream inside a conduit, to the point that it is not certain that the functional component is supplied with a sufficient oil flow rate so that the measurements which it carries out are correct.

WO-A-03/091550 discloses a method for analyzing a lubricant in which a measurement, carried out by means of an XRF sensor on a sample of a lubricant to be monitored, is compared with measurements carried out on reference lubricant samples. This approach is provided for an operation in a laboratory and requires qualified labour.

Moreover the use of a spectrometer comprising an X-ray source and a X-ray detector for measuring the metal particle content in a lubricant due to the wear of the lubrication system of an engine is known from U.S. Pat. No. 5,982,847. This X-ray source and this detector are associated with a cell made in a non-metal material or based on aluminium and which is equipped with a transparent window to X-rays and which may be made for example in beryllium, in boron or in a polymer coated with aluminium. The beryllium gives satisfactory results but may prove to be toxic for an operator. The other materials tend to filter the X-rays. Considering the pressure and the temperature of the fluid circulating in the cell, the window has to be relatively thick, in order to resist to mechanical forces which it undergoes but this has the effect of attenuating the radiation which crosses it, both between the X-ray source and the sample to be analyzed and between this sample and the radiation detector.

On the other hand, the equipment of laboratories, such as the one known from U.S. Pat. No. 6,233,307 which may allow the detection of the dissolved iron content in a lubricant, are difficult to transport and of a complex use, which makes them not very accessible to the seagoing personnel, even trained for this.

These problems are posed not only on two-stroke or four-stroke engines for propelling ships, but also on other secondary engines also onboard the ships, for example for accessories of the hoist type. Generally, the monitoring of the overall iron content, i.e. the dissolved and/or particulate iron content, in a lubricant is important for all the lubricated engines and the known techniques are not very favorable for automation.

Similar problems are posed for the determination of the content of a lubricant in another predetermined chemical element, notably in calcium, vanadium, chromium, molybdenum, copper, sulfur, lead, silver, tin, aluminium, nickel, zinc or phosphorus.

It is to these drawbacks which the invention more particularly intends to remedy by proposing a novel installation for following up the evolution of the quality of a lubricant circulating in a piece of equipment which is adapted for operating in a simple and autonomous way, which notably discharges the personnel onboard a ship from repetitive and elaborate tasks.

For this purpose, the invention relates to an installation for following up the evolution of the quality of a lubricant circulating in a piece of equipment, this installation comprising at least one conduit for circulating the lubricant, this conduit being connected, upstream to the piece of equipment and downstream to a recovery pan. This installation further comprises a sensor for determining, by the X fluorescence technique, the overall content in a predetermined chemical element, of a lubricant sample, this sensor comprising an X-ray source, an X-ray detector and a cell intended to contain a lubricant sample to be analyzed and with a wall forming a window for letting through the X-rays stemming from the source or going towards the X-ray detector. According to the invention, this installation comprises a first controlled interruption valve for the circulation of the lubricant in the conduit, a buffer reservoir for accumulating lubricant, a first bypass line connected on the one hand to the conduit, upstream from the first valve and on the other hand to the buffer reservoir, a second controlled interruption valve for the circulation of the lubricant in the first bypass line, a second line for discharging the lubricant, from the buffer reservoir to the recovery pan, as well as a third controlled interruption valve for the circulation of the lubricant in the second discharge line. The sensor determines the overall content in a predetermined chemical element of a lubricant sample at the output of the buffer reservoir. Further, the wall which forms a window for letting through the X-rays is made in poly(ethylene terephthalate).

By means of the invention, the sensor which operates with the X fluorescence technique allows an onboard measurement of the overall content in a predetermined chemical element in a lubricant. In the sense of the present invention, the overall content in a chemical element in a lubricant is the content in the dissolved and particulate element of this chemical element in this lubricant. Advantageously, the sensor is a sensor for the iron content in the lubricant. The measurement made is not affected, substantially by the wall which forms the window for letting through the X-rays. Indeed, this wall made in poly(ethylene terephthalate) or PET, may be provided with a relatively not very large thickness, notably less than 200 μm (micrometer), while having satisfactory mechanical properties for resisting to the pressure forces and vibrations within an installation according to the invention. The constitutive material of the wall also allows it to not be altered by the lubricant which circulates in the cell.

The lubricant which is relevant in the present invention is a lubricant conventionally used and known by one skilled in the art and which allows lubrication of a piece of equipment, notably a ship engine. This lubricant comprises at least one lubricating base oil. Generally, lubricating base oils may be oils of mineral origin, synthetic or vegetable oils as well as their mixtures.

In the lubricants, the lubricating base oils may be used alone or as a mixture. For example, a mineral oil may be combined with a synthetic oil. The lubricant may further comprise at least one additive conventionally used and known to one skilled in the art for formulating a lubricant and more particularly a marine lubricant. As an example, the additive may be selected from among overbased detergents, neutral detergents, dispersants, anti-wear additives, any other functional additive, or mixtures thereof.

For example, the overbased detergent and the neutral detergent may be selected from among carboxylates, sulfonates, salicylates, naphthenates, phenates, and the mixed detergents associating at least two of these types of detergents. The overbased detergent and the neutral detergent are notably compounds based on metals selected from among calcium, magnesium, sodium or barium, preferentially calcium or magnesium. The overbased detergent may be overbased with insoluble metal salts selected from the group of alkaline metal and earth-alkaline metal carbonates, preferentially calcium carbonate. The lubricant may comprise at least one overbased detergent and at least one neutral detergent as defined above.

For example, the compounds derived from succinic acid are dispersants particularly used as lubrication additives. Mannich bases, obtained by polycondensation of phenols substituted with alkyl groups, with formaldehyde and primary or secondary amines, are also compounds used as dispersants in lubricants.

As an example, from among the anti-wear additives conventionally used for formulating marine lubricants, the most currently used anti-wear additive is zinc dithiophosphate or DTPZn.

For example, the other functional additives may be selected from among thickeners, anti-foam additives for acting against the effect of the detergents, which may for example be polar polymers such as polymethylsiloxanes, polyacrylates, anti-oxidant and/or anti-rust additives, for example organo-metal detergents or thiadiazoles.

According to advantageous but non-mandatory aspects, an installation according to the invention may incorporate one or several of the following features, taken in any technically admissible combination:
The cell comprises a metal box on which the wall is added.
The cell comprises a hollow housing for receiving the wall and a threaded washer for immobilizing the wall in the hollow housing.
The sensor is a sensor for determining the iron content of the sample of lubricant.
The casing is made in a metal or a non-ferrous alloy, notably in an alloy based on aluminium.
The X-ray source and the X-ray detector are mounted on a lid which position them relatively to the casing and to the wall, while this support forms a shield against the propagation of X-rays.
An axis for targeting the X-ray source and an axis for targeting the X-ray detector form between them an angle comprised between 20° and 25°, preferably of the order of 22°.
The wall has a thickness of less than or equal to 200 μm, preferably less than or equal to 150 μm, still preferably of the order of 125 μm.
The sensor for determining the content in a predetermined chemical element is positioned on the second discharge line.
The installation further comprises a sensor for determining the basicity index of the lubricant, positioned on the second discharge line and which allows determination of the basicity index of the lubricant at the output of the buffer reservoir.

Moreover, the invention relates to an automated method for following up the evolution of the overall content in a predetermined chemical element in a lubricant circulating in a piece of equipment, by means of an installation as mentioned above, wherein the sensor for determining the iron content is positioned on the second discharge line. This method is characterized in that it comprises at least steps consisting of:
a) closing the first valve,
b) opening the second valve and closing the third valve in order to supply the buffer reservoir from an amount of accumulated lubricant in the conduit, upstream from the first valve,
c) opening the third valve in order to circulate the lubricant present in the buffer reservoir through the second discharge line as far as into a cell of the sensor,
d) using the sensor for determining the overall content in a predetermined chemical element of the lubricant at the output of the buffer reservoir.

The invention also relates to a method for tracking the operation of a piece of equipment loaded onboard a ship, this method comprising the determination, onboard the ship, of the overall content in a predetermined chemical element, notably in iron, of the lubricant circulating in the relevant piece of equipment by applying an automated method as mentioned above.

Finally, the invention relates to the use of an automated method as mentioned above for determining the overall content in a predetermined chemical element, notably in iron, of a lubricant circulating in a piece of equipment of a ship, in particular in a ship engine.

The invention will be better understood and other advantages thereof will become more clearly apparent in the light of the description which follows of two embodiments of an installation according to its principle, exclusively given as an example and made with reference to the appended drawings wherein:

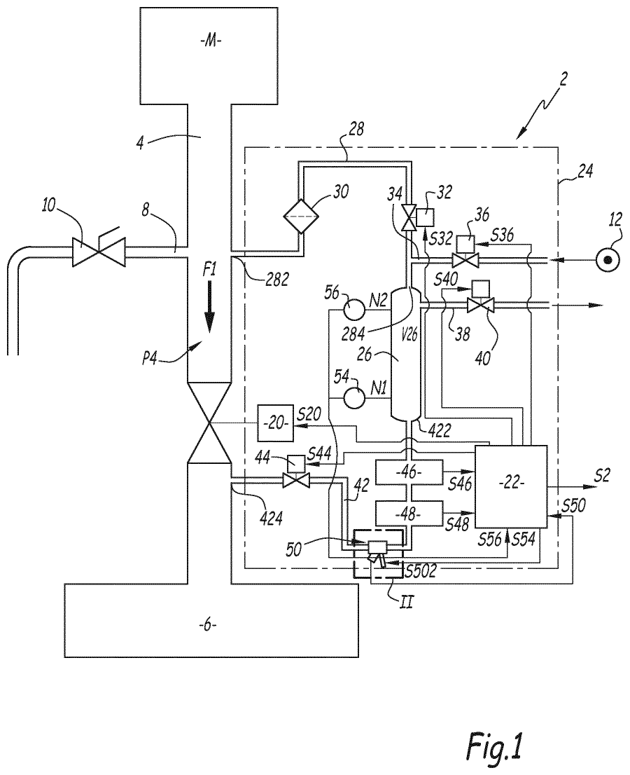

FIG. 1 is a schematic representation of an installation according to the invention as loaded onboard a ship,

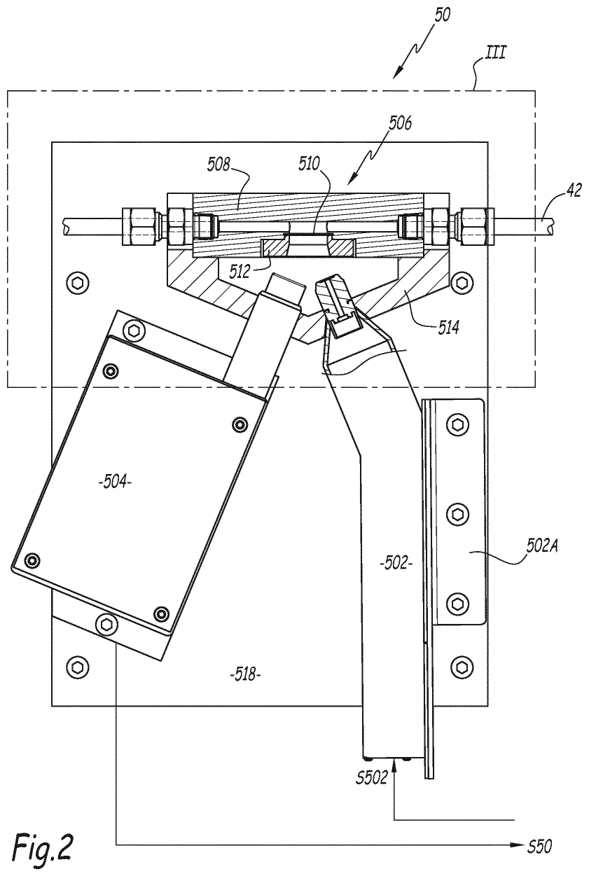

FIG. 2 is a view at a larger scale of the detail II in FIG. 1, showing a sensor for determining the overall content in a predetermined chemical element, notably in iron, used in the installation of FIG. 1.

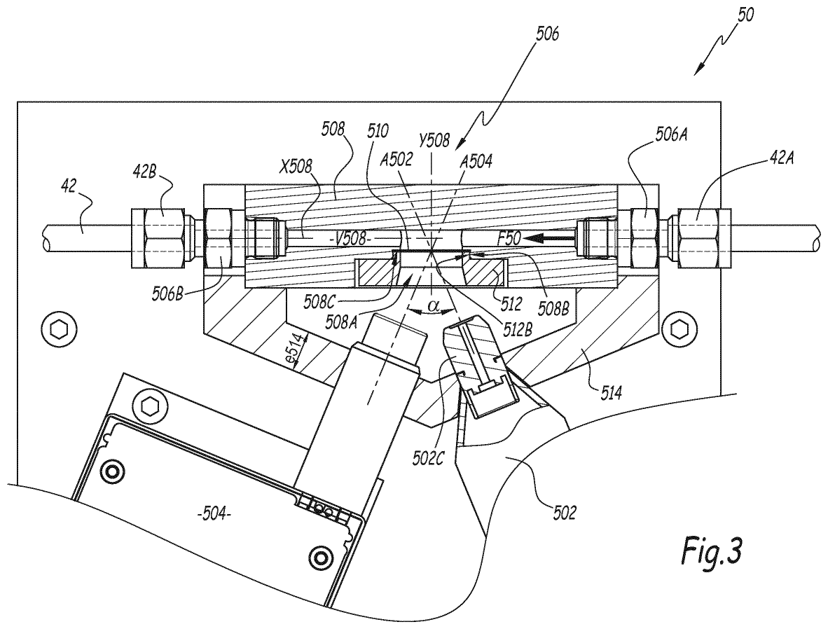

FIG. 3 is a view at a larger scale of the detail III in FIG. 2,

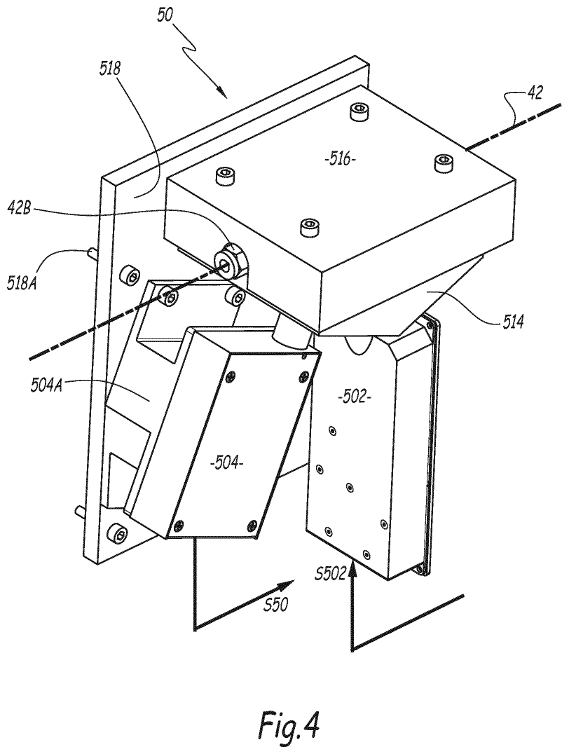

FIG. 4 is a perspective view of the sensor illustrated in FIGS. 2 and 3,

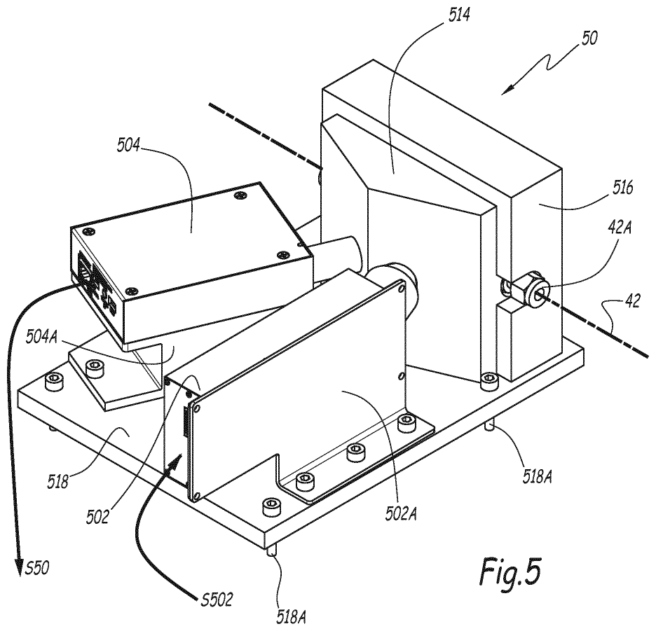

FIG. 5 is a perspective view according to another angle, of the sensor of FIGS. 2 to 4,

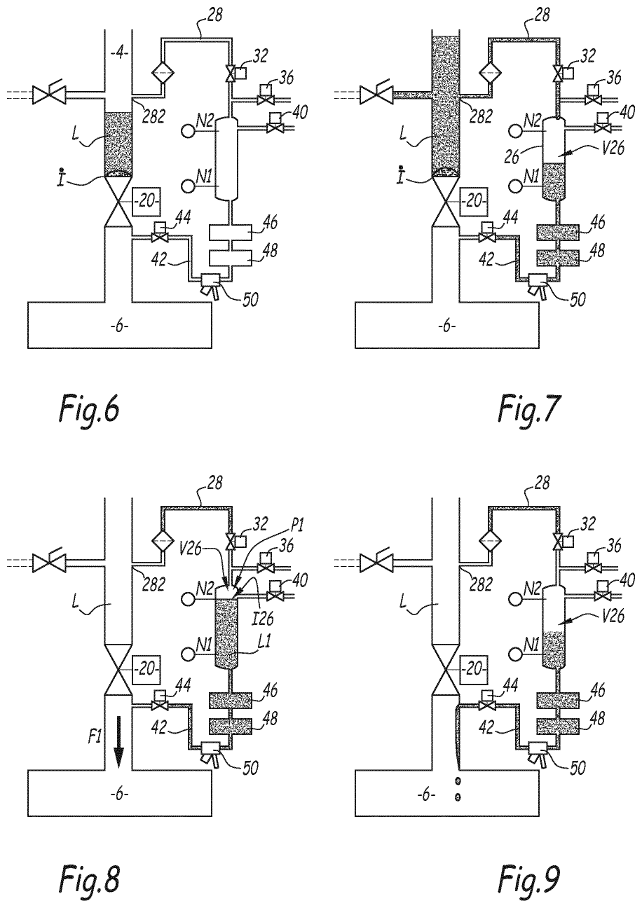

FIG. 6 is a schematic illustration at a smaller scale of the fluidic portion of the installation of FIG. 1 in a first configuration of use,

FIGS. 7 to 9 are similar views to FIG. 6 when the installation is in a second, a third and a fourth configuration for use,

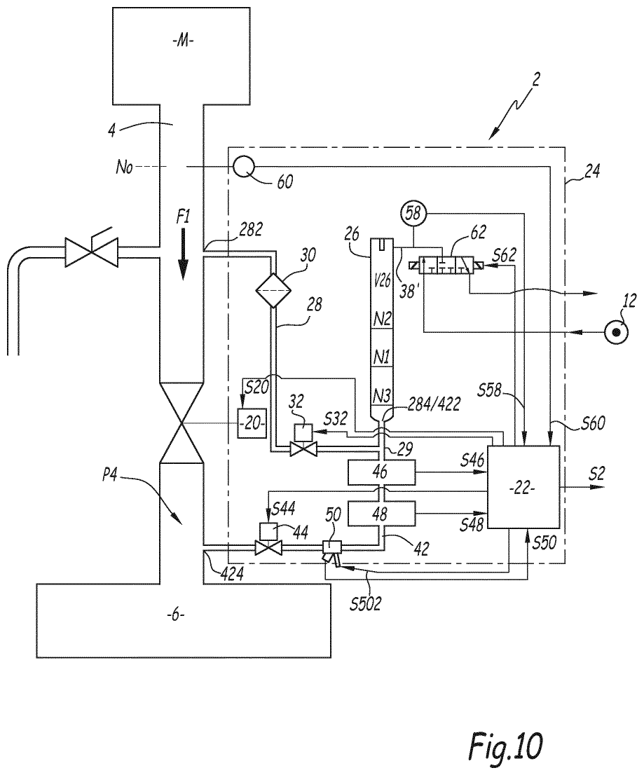

FIG. 10 is a similar view to FIG. 1 for an installation according to a second embodiment of the invention,

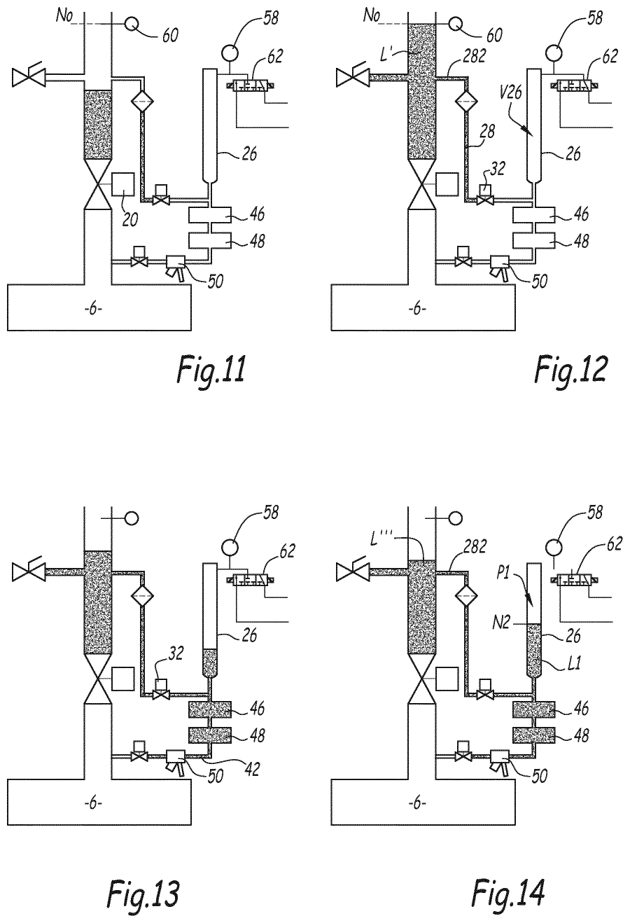

FIGS. 11 to 21 are similar views to FIG. 6 for the installation of FIG. 10 in various configurations for use, and

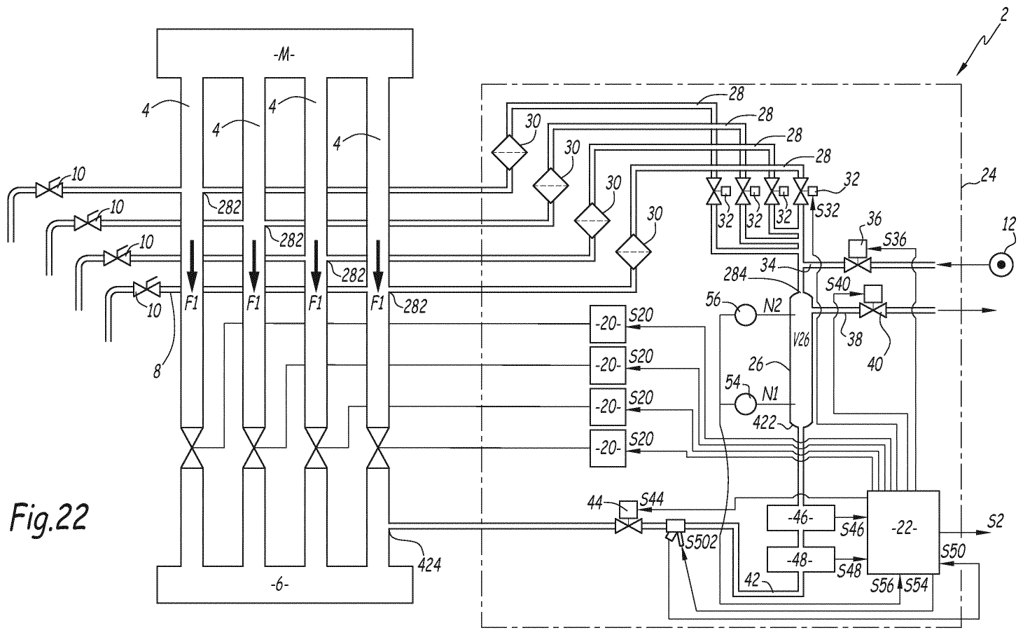

FIG. 22 is a view similar to FIG. 1 for an installation according to a third embodiment of the invention.

In FIGS. 6 to 9 and 11 to 21, the lubricant present or circulating in a portion of the installation is illustrated as a shaded illustration.

The installation 2 illustrated in FIGS. 1 to 9 is loaded onboard a ship illustrated in FIG. 1 by a piece of equipment M. In particular, the piece of equipment M is an engine which includes several cylinders, for example from two to fourteen cylinders, preferably a two-stroke or four-stroke engine of the ship. A conduit 4 connects the piece of equipment M to a pan 6 for recovering lubricant. In practice, the engine oil flows in the conduit 4 with a pressure P4 comprised between 1.1 and 6 absolute bars. The oil flow rate in the conduit 4 may be low, to the point that the oil trickles on the internal wall of this conduit.

The conduit 4 extends vertically, from top to bottom, of the piece of equipment M towards the pan 6. In this embodiment, the oil flowing in the conduit 4 comes from at least one cylinder of the piece of equipment M.

A tapping connection 8 is provided on the conduit 4 and equipped with a valve 10 manually controlled, which gives the possibility of sampling an amount of oil leaving the piece of equipment M in order to proceed with physico-chemical analyses, according to an approach known per se.

The installation 2 comprises a stop valve 20 mounted on the conduit 4 and which gives the possibility of interrupting, selectively, the flow of oil into the conduit 4, towards the pan 6. The stop valve 20 is controlled by an electronic unit 22 by means of an electrical signal S20.

As exclusively visible in FIG. 1, the installation 2 comprises a box 24 illustrated by its trace in an axis line and inside which are positioned the constitutive elements of the installation 2, except for the portion of the stop valve 20 which is integrated to the conduit 4.

The installation 2 also comprises a buffer reservoir 26 which is positioned in the box 24 and which is connected to the conduit 4 by means of a first bypass line 28.

The mouth of the line 28 is noted as 282. This mouth is positioned upstream from the valve 20 on the conduit 4. The first bypass line 28 is equipped, downstream from its mouth 282 towards its opening 284 into the buffer reservoir 26, with a filter 30, with a stop valve 32 and with a tapping connection 34. The filter 30 is used for preventing impurities of a too large size from flowing into the first bypass line 28. The stop valve 32 gives the possibility selectably, of unblocking or closing the first bypass line 28. The valve 32 is controlled by the electronic unit 22, by means of an electrical signal S32. The tapping connection 34 is connected, through a control valve 36, to a source 12 of pressurized air which is not part of the installation 2 but belongs to standard equipment of a ship.

In practice, the source of pressurized air 12 may be a compressor loaded onboard the ship and which supplies a compressed air network which is also used with pieces of equipment other than the installation 2. Alternatively, the source 12 may be a pump dedicated to the installation 2.

The installation 2 also comprises a tapping connection 38 connected to the reservoir 26, on which is mounted a stop valve 40 and which gives the possibility of allowing the inner space V26 of the reservoir 26 communicate with the ambient atmosphere.

In this embodiment, the tapping connections 34 and 38 are independent. Alternatively, they may be replaced with a single tapping connection, connected to the first line 28 or directly to the reservoir 26, on which the valves 36 and 40 are mounted in parallel, while being respectively connected to the pressurized air source 12 and to the ambient atmosphere. In this case, it is possible to combine the valves 36 and 40 in the form of a single three-way valve.

The valves 36 and 40 are controlled by the electronic unit 22 by means of respective electric signals S36 and S40.

The installation 2 also comprises a second discharge line 42 for the lubricant, from the inner space V26 of the reservoir 26 towards the recovery pan 6. The second discharge line 42 is therefore positioned downstream from the first bypass line 28 and from the reservoir 26, on the flow path of the lubricant. In the example, the second line 42 extends from the reservoir 26 towards the conduit 4. Its mouth 422 is located in the low portion of the reservoir 26, while its opening 424 is positioned on the conduit 4, downstream from the stop valve 20, as illustrated in the figures, which gives the possibility of reducing the time of an analysis cycle since the stop valve 20 may be closed in order to generate a column of oil in the conduit 4, while measurement steps take place. Alternatively, the opening 424 of the second line 42 is positioned upstream from the stop valve 20, which gives the possibility of simultaneously executing steps for emptying and unblocking the filter 30 and optionally reducing the cost of the installation 2.

The second line 42 is equipped with a stop valve 44 which is controlled by the electronic unit 22 by means of an electric signal S44.

Three sensors 46, 48 and 50 are positioned on the line 42, upstream from the valve 44.

The sensor 46 gives the possibility of measuring the density D, the viscosity V, the humidity H and the temperature T of a lubricant, present or flowing in the second line 42. This sensor may be of the type of the one marketed by AVENISENSE under the name of Cactus. Alternatively, the sensor 46 may be of another type and only allows measurement of a single one or some of the parameters mentioned above.

The sensor 48 is a sensor of a basicity index or BN, sometimes called an alkalinity index. This may be a sensor operating with infrared technology, in the infrared means, or of any other sensor suitable for determining the BN of a lubricant.

The sensor 50 is a sensor for determining the overall iron content, i.e. the content of dissolved and/or particulate iron, of a lubricant sample leaving the reservoir 26, by means of X fluorescence technology.

As this more particularly emerges from FIGS. 2 to 5, the sensor 50 comprises an X-ray source 502, an X-ray detector 504 and a cell 506 which is mounted in series on the second line 42. To do this, the cell 506 is provided with an upstream connecting element 506A which cooperates with the complementary connecting element 42A provided on line 42, as well as with a downstream connecting element 506B which cooperates with a complementary connecting element 42B provided on line 42.

The source 502 comprises a cathode and an anode between which circulate electrons, under the effect of a potential difference of the order of 50 kV, a current of the order of 500 mA circulating in the cathode. The anode is made in metal, for example in gold, tungsten, silver or rhodium. The power required for the source 502 is relatively moderate, notably comprised between 4 and 10 W. A collimator 502C is used at the outlet of the source 502 for concentrating the electron beam centered on a targeting axis A502 of the source 502.

The rays emitted by the X source are in the range of X-rays, with a wavelength comprised between 0.01 and 10 nm, i.e. with a frequency comprised between about 3×10¹⁹ and 3×10¹⁶ Hz.

The detector 504 is of the SDD (Silicon Drift Detector) type which comprises a single electrode on the front face, which collects the electrons generated by the interaction of the X-rays in the pn junction of a photo diode. This type of detector has the advantage of a low capacitance due to the small surface of its anode. This type of detector gives the possibility of obtaining a high counting rate, good resolution and efficient cooling with the Peltier effect. Alternatively, the detector 504 is of the SI-PIN type, with a silicon photo diode, which has an intrinsic zone inserted between two respectively positively and negatively doped zones.

The detector 504 is capable of counting the emission «counts» at each energy level over a given period, which allows the establishment of a spectrum of energy levels. The targeting axis of the detector 504 is noted as A504, which corresponds to the main direction of the X-rays detected by this detector.

The cell 506 comprises a body 508 made by machining or molding a block of metal. This body 508 is preferably made in aluminium or in an alloy based on aluminium, for example Zicral (7075) which is an aluminium alloy with zinc as the main alloy element. Alternatively, the casing 508 may be made in another alloy based on aluminium. In the sense of the present description, an alloy based on aluminium is an alloy which comprises at least 50% by weight of aluminium. The use of an alloy based on aluminium allows the casing 508 to resist to temperature, to pressure and to the chemical composition of the lubricant flowing in the line 42. The absence or the low proportion of iron contained in this alloy avoids that the measurement by X fluorescence of the overall iron content of the lubricant be perturbed by the presence of iron in a significant amount.

However, alternatively, it is possible to provide that the casing 508 is made in stainless steel.

The casing 508 defines a volume V508 for circulation of the lubricant between the connecting elements 506A and 506B, in the direction of the arrow F50 in FIG. 3. This volume V508 has a tubular shape with a circular or rectangular section, chosen by the designer of the casing 508. A longitudinal axis of the volume V508 is noted as X508.

The casing 508 is provided with a pierced hole 508A, which is centered on an axis Y508 perpendicular to the axis X508 and which opens into the volume V508. The pierced hole 508A is with a circular section centered on the axis Y508 and provided with a tapped hole 508B.

The pierced hole 508A is obturated with a wall 510 with the shape of a disc, which may also be described as a «membrane» and which is maintained pressed against the bottom of a counter-bore 508C of the pierced hole 508A by means of a ring 512 provided with external threading 512B mating the tapping 508B and which cooperates with the latter. The wall or membrane 510 is mounted on the casing 508 by flattening it against the counter-bore 508C, and then by screwing the ring 512 into the pierced hole 508A.

The ring 512 is made in the same metal or the same alloy as the body 508.

The wall 510 forms a target window for the source 502 and for the detector 504 which allows the X-rays to pass in transit from the source 502 to a lubricant sample contained in the volume V508 and from the volume 508 to the detector 504.

The wall 510 is made in poly(terephthalate) or PET, which gives it satisfactory mechanical properties, while it may have a small thickness, of less than 200 μm, to the point that it does not perturb the X-rays which stem from the source 502 or which leave towards the detector 504.

In practice, the thickness of the wall 510, which is measured perpendicularly to the axis Y508, may be selected to be less than 150 μm, preferably of the order of 125 μm.

The source 502 and the detector 504 are mounted on a lid 514 which determines their position relatively to the cell 506, more particularly relatively to the casing 508 and to the wall 510. This lid 504 surrounds the casing 508 on the side of the pierced hole 508A, so that it insulates the window formed by the wall 510 from the outside of the sensor 50. The thickness of the 514 is noted as e514. The material of this lid 514 and its thickness e514 are selected in such a way that they form an efficient shield against the X-rays which circulate between the source 502, the detector 504 and the cell 506. This cell allows free passing of the lubricant and gives the possibility of carrying out static or dynamic analyses of the fluid lubricant. In practice, the lid 514 may be made in stainless steel, for example of the 316 type, and the thickness e514 is selected to be greater than 5 mm, preferably greater than 8 mm, still preferably of the order of 10 mm.

On the other hand, a complementary shield 516 is mounted around the casing 508, on the side of this casing opposite to the lid 514. For the clarity of the drawing, this shield 516 is exclusively illustrated in FIGS. 4 and 5.

The portions 502, 504, 506, 514 and 516 of the sensor 50 are attached on a support 518 formed by a plate which may be immobilized in the box 24 by means of screws 518A. A square 502A and a spacer 54A are used for respectively attaching the source 502 and the detector 504 on the support 518.

Thus assembled, the sensor 50 is a sub-assembly easy to handle, easily identifiable, and which may be the object of a standard exchange operation, by unscrewing the screws 518A and by separating the connecting elements 506A and 42A, on the one hand, 506B and 42B, on the other hand.

The source 502 is controlled by the electronic unit 22 by means of a signal S502 and the detector 504 delivers to the electronic unit 22 an output signal S50 of the sensor 50.

In practice, the axes X508, Y508, A502 and A504 are coplanar.

An angle measured between the axes A502 and A504 outside the casing 508 is noted as α. This angle α has a value comprised between 20 and 25°, preferably of the order of 22°.

During operation, the X fluorescence technology of the sensor 50 is used for determining the overall iron content, i.e. the content of dissolved and/or particulate iron, of an amount of oil passing in transit through the volume V508, at the outlet of the buffer reservoir 26.

The measurement of the iron content carried out by the sensor 50 when the lubricant flows into the second line 42, i.e. when the valve 44 is open. This is then referred to as a dynamic measurement.

Alternatively, this measurement may be carried out when the lubricant is static in the volume V508, i.e. when the valve 44 is closed. This is then referred to as a static measurement.

The installation 2 also comprises a first level sensor 54 and a second level sensor 56 which respectively allows detection when the amount of oil in the reservoir 26 reaches a first level N1 or a second level N2. The electric output signals S54 and S56 of the sensors 54 and 56 are delivered to the unit 22.

Alternatively, the sensors 54 and 56 may be replaced with a single sensor, such as a pressure sensor, which allows detection when the oil reaches each of the two levels N1 and N2 in the reservoir 26.

FIGS. 6 to 9 schematically illustrate the successive steps of an automated method applied by means of the installation 2 of FIG. 1. This method is automated in the sense that it may be implemented, either partly or preferably totally, without any human intervention, under the control of the unit 22. The same applies for the method explained hereafter regarding the second embodiment of the invention.

By default, and outside the sampling phases, the oil leaving the piece of equipment M flows into the conduit 4, in the sense of the arrow F1 in FIG. 1, from the piece of equipment M to the recovery pan 6, without being retained by the valve 20 which is in an open or unblock configuration, while the other valves are closed.

When the iron content of the oil leaving the engine M should be determined, the unit 22 controls the valve 20 upon closing, so that a retention is generated in the conduit 4 where an oil amount accumulates, i.e. of lubricant, as illustrated in the shaded portion L in FIG. 2.

In the configuration of FIG. 6, the conduit 4 is used as a decantation column and impurities I accumulate in the vicinity of the valve 20, inside the conduit 4 and in the lower portion of the amount of lubricant L.

In this first step illustrated by the configuration of FIG. 6, the valves 32 and 40 are open, while the valves 36 and 44 are closed.

When the lubricant level L in the conduit or column 4 attains the mouth 282, oil begins to flow through the first bypass line 28, more particularly through the filter 30 and the valve 32, as far as into the inner volume V26 of the reservoir 26 in which the oil flows. Indeed, the opening 284 of the first line 28 is located in the upper portion of the reservoir 26 and the oil may flow along the wall of the reservoir 26. As the valve 44 is closed, the oil gradually fills up the portion of the second discharge line 42 located upstream from the valve 44, including the internal volumes of the sensors 46 and 48, and then the inner volume V26, by driving the air to the atmosphere, through the valve 40. This step corresponds to the configuration illustrated in FIG. 7.

When the sensor 56 detects that the oil level N2 inside the reservoir 26 is attained, the unit 22 switches the installation 2 to a new configuration illustrated in FIG. 8, wherein the valve 20 passes into the open configuration, which gives the possibility of emptying the decantation column by directing the remainder of the lubricant amount L present upstream from the valve 20 as well as the impurities I towards the recovery pan 6. The flow in the direction of the arrow F1 therefore continues as far as into the pan 6. Moreover, the valves 32 and 40 are closed and the valve 36 is open, which gives the possibility of setting the portion of the volume V26 which is not occupied by the lubricant, i.e. the portion of this volume V26 located above the level N2, under an air pressure P1 equal to that of the air source 12, which in the example, has the value of 7 absolute bars.

This being done, the unit 22 has the installation 2 pass to a next step, illustrated by the configuration of FIG. 9, wherein the valve 44 is open, the other valves retaining their configuration state of FIG. 4. In this case, the pressure P1 of the air in the upper portion of the volume V26 has the effect of pushing the oil into the second discharge line 42, through the sensors 46, 48 and 50, which allows these sensors to provide the unit 22 with signals S46, respectively S48 and S50, representative of the parameters which they have detected.

If necessary, the signals S46, S48 and S50 may be processed in the unit 22 in order to determine the values of the controlled parameters, notably by comparison with values known for reference lubricants.

The signals S46, S48 and S50, or signals extrapolated from these signals, may be provided at the outside of the installation 2 in the form of a conjugate signal S2, utilizable by a central unit for monitoring the piece of equipment M.

In practice, the passage section of the iron content sensor 50 is of about 70 mm². It may range up to 200 mm². In every case, this passage section should be able to be supplied with a sufficient flow, for a sufficient time for carrying out the measurement of the overall iron content. Alternatively, the same applies for the sensor 46 and for the sensor 48 of the basicity index. The design of the installation with the reservoir 26 gives the possibility of generating a reserve forming a «buffer» of oil, as the amount of oil L1 contained in the reservoir 26 in the configuration of FIG. 4. A portion of this oil reserve L1 may be poured, either continuously or sequentially, into the second discharge line 42 so that the sensors 48 and 50 have a sufficient amount of oil to be analyzed.

From the configuration of FIG. 9, it is possible in a subsequent step to continue the emptying of the reservoir 26 and of the integrality of the second discharge line 42 by maintaining the valve 44 open and by continuing the injection of compressed air through the valve 36.

Alternatively, it is possible to stop the emptying of the reservoir 26 when the oil level attains the level N1, so as to permanently retain an amount L2 of oil in the second discharge line 42, in particular in the sensors 46, 48 and 50 for which the active portions in contact with oil do not risk drying. This notably avoids the deposition of oil traces on the wall 510 of the sensor 50. If this second approach is selected, a certain amount of oil has to be used during a next measurement, for cleaning beforehand the second discharge line 42 and not perturbing the next measurement.

In the second and third embodiments of the invention illustrated in FIG. 10 and the following, the elements similar to those of the first embodiment bear the same references. In the following, one mainly describes what distinguishes these embodiments from the preceding one.

In the embodiment of FIGS. 10 to 21, the first and second lines 28 and 42 join up at a T junction 29. Thus, the opening 284 of the first discharge line 28 coincides with the mouth 422 of the second discharge line 42. The line segment located between the reservoir 26 and the junction 29 is common to the first and second lines 28 and 42. This line segment opens into the low portion of the reservoir 26, so that the oil which flows from the conduit 4 towards the reservoir 26 directly reaches the low portion of this reservoir.

Three levels N1, N2 and N3 are defined in the reservoir 26, the levels N1 and N2 being comparable with those of the first embodiment.

In this second embodiment, no level sensors identical with the level sensors 54 and 56 are not used, but a pressure sensor 58 for which the output signal S58 is provided at the electronic control unit 22. Moreover, a level sensor 60 is mounted in the conduit 4, upstream from the valve 20, i.e. above the latter. It provides the unit 22 with a signal S60.

Further, the tapping connections 34 and 38 and the valves 36 and 40 of the first embodiment are replaced with a single tapping connection 38′ on which is connected the pressure sensor 58, as well as a three-way distributor 62 and three orifices, which is connected on the one hand to the source of pressurized air 12 and on the other hand to the ambient atmosphere. The distributor 62 is controlled by the unit 22 by means of a dedicated electric signal S62.

The installation 2 comprises, like in the first embodiment, a sensor 50 for determining the overall iron content, i.e. the content of dissolved and/or particulate iron, identical with that of the first embodiment and mounted on the conduit 42.

The operation of the installation 2 is the following:

By default, the valve 20 is open and the valves 32 and 44 are closed, while the distributor 62 is in the configuration illustrated in FIG. 10 where it isolates the inner volume V26 of the reservoir 26 of the compressed air source 12 and from the ambient atmosphere.

When one should proceed with the determination of the iron content and optionally of the basicity index of the oil leaving the piece of equipment M, the unit 22 activates the valve 20 by means of the signal S20 in a first step, in order to bring it into the closed configuration illustrated in FIG. 11. In this configuration, oil is present in the first bypass line 28, between the filter 30 and the valve 32, because of an unblocking operation of the filter 30, carried out previously and which is explained hereafter.

In this configuration, the valves 32 and 44 and the distributor 62 are closed.

The level sensor 60 is positioned so that, when the oil column retained in the conduit 4 upstream from the valve 20 reaches the level NO detected by this sensor, as illustrated in FIG. 12, a predetermined amount of lubricant is present above the opening 282. For example, the predetermined amount may be equal to 100 ml. When the level sensor 60 detects that this level NO is attained in the conduit 4, the inner volume V26 of the reservoir 26 is set to atmospheric pressure by actuating the distributor 62 for bringing it into the configuration of FIG. 12.

From this configuration, the unit 22 controls the valve 32 and the distributor 62 in a next step for carrying out the transfer of the amount of oil from the conduit 4 to the reservoir 26, as illustrated by the configuration of FIG. 13. In this configuration, the valve 32 is open, while the distributor 62 is closed. The transfer of oil from the conduit 4 to the buffer reservoir 26 is therefore accompanied by an increase in the air pressure inside the reservoir 26. The compression level of the air confined in the reservoir may be connected, after calibration, to the initial volume of air in the reservoir 26 and to the volume of transferred oil.

For example, for an adiabatic compression and an initial air volume in the reservoir 26 equal to 160 ml, the pressure in the reservoir 26 attains 1.7 absolute bars for 50 ml of transferred oil.

Also, by considering a reservoir 26 containing initially 250 ml of air, it is possible to transfer 80 ml, i.e. the amount L1 illustrated in FIG. 10, into the reservoir 26 before attaining in the upper portion of the latter an air pressure of P1 equal to 17 absolute bars. This is the considered example in the following.

In this case, the oil level N2 is attained in the reservoir 26 at the step represented by the installation 2 in the configuration of FIG. 14.

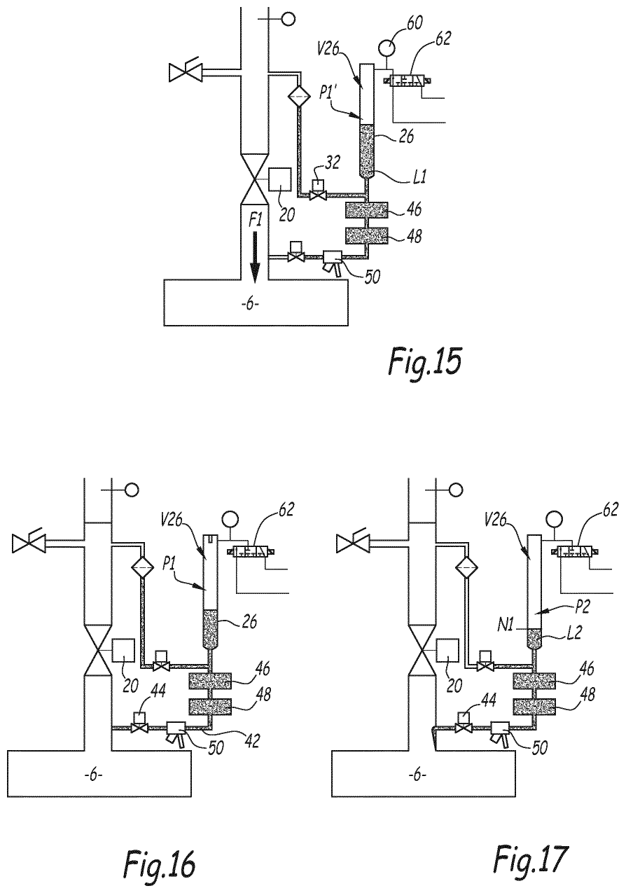

The unit 22 then automatically controls the valves and the distributor for attaining the configuration of FIG. 15 wherein the reservoir 26 is pressurized through the distributor 62 which connects the volume V26 to the source of compressed air 12, so that the air pressure P1′ inside the reservoir 26 becomes equal to 7 bars. In order that this may occur, the valve 32 was switched beforehand by the unit 22 into the closed configuration, in order to avoid a return of oil from the reservoir 26 to the conduit 4. Moreover, in this step, the valve 20 is switched by the unit 22 into the open configuration, so that the flow of engine oil circulating in the piece of equipment M towards the recovery pan 6 may again take place in the direction of the arrow F1. From this configuration, the unit 22 controls the distributor 62 and the valve 44 by means of the signals S62 and S44, respectively for closing the distributor 62 and opening the valve 44 and thereby attaining the configuration of FIG. 16 wherein the oil contained in the reservoir 26 is gradually driven from the latter because of the pressure P1 prevailing in the upper portion of the inner volume V26.

The oil therefore flows through the sensors 46, 48 and 50 which are capable of detecting the parameters for which they are provided and of providing the corresponding signals, S46, S48 and S50, to the unit 22, like in the first embodiment.

The discharge of oil contained in the reservoir 26 through the second discharge line 42 may take place in several cycles, by successive expansions of the confined air volume in the reservoir and successive connections to the air source 12. For a reservoir of 250 ml initially containing 80 ml of oil, it is for example possible to perform three successive expansions, between 7 bars and 6.2 bars, preceded with three connections to the air source 12. This gives the possibility of delivering a total volume of 50 ml into the second discharge line 42 and to attain the configuration of FIG. 17 wherein a residual amount L2, of 30 ml of lubricant, remains in the reservoir 26 while being subject to a pressure P2 equal to 6.2 bars.

The three successive expansions take place by filling beforehand and successively the reservoir 26 with air at 7 bars, by means of a suitable command of the distributor 62.

These three expansions give the possibility of circulating 50 ml of lubricant in the sensors 46, 48 and 50 in three successive steps, which allows them to generate three sets of signals S46, S48 and S50 or a set of combined signals, intended for and provided for the unit 22, and then transmitted and/or processed like in the first embodiment.

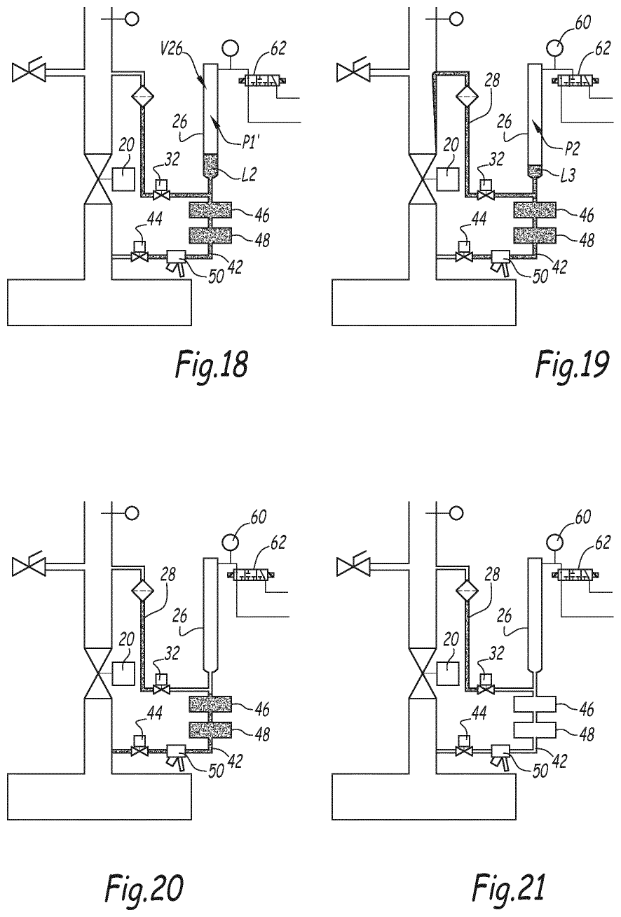

From the configuration of FIG. 17, the unit 22 causes the installation 2 to pass into the configuration of FIG. 18 wherein the inner volume V26 of the reservoir 26 is again pressurized to the pressure P1′ of 7 bars, in return for a suitable command of the distributor 62, while the valve 44 is closed.

Once this operation is completed, the unit 22 controls the valve 32 to open and the distributor 62 to close, which has the effect of driving the oil present in the low portion of the reservoir 26 through the first bypass line 28, as far as into the conduit 4, in a direction for unblocking the filter 30. This step is illustrated by the configuration of FIG. 19. The fact of lowering the pressure in the reservoir 26 from 7 to 6.2 bars gives the possibility of having an amount of about 20 ml circulate from the reservoir 26 to the conduit 4. At the end of this step, there remains an amount L3 equal to 10 ml of lubricant in the reservoir 26, under a pressure P2 of 6.2 bars.

Once this unblocking operation of the filter is completed, the unit 22 has the installation 2 pass into the configuration of FIG. 20 wherein the valve 32 is again closed, while the valve 44 is open and the distributor 62 is placed in a configuration for supplying the volume V26 with pressurized air. This has the effect of discharging the residual amount of oil present in the second discharge line 42 and in the sensors 46 and 48 until the configuration of FIG. 21 is obtained wherein the second discharge line 42 and the sensors 46 and 48 are empty of oil and filled with air. This corresponds to the configuration of FIG. 11 mentioned above.

It is noted that in the FIGS. 20 and 21 the portion of the first bypass line 28 located between the valve 32 and the opening 284 is emptied by the air stemming from the reservoir 26. This is to be conciled with the fact that in practice, the valve 32 is immediately positioned downstream from the junction 29.

In the third embodiment of the invention illustrated in FIG. 22, first conduits 4 are used, each of them being provided for collecting the oil from a single cylinder of the piece of equipment M.

Each conduit 4 is equipped with a valve 20 controlled by the electronic unit 22 and which allows interruption of the stream of a flow F1 of lubricant in the relevant conduit 4. A first bypass line 28 is connected on the one hand to each conduit 4, upstream from its valve 20 and on the other hand at the inlet of the buffer reservoir 26 which is the same as in the first embodiment. The installation 2 therefore comprises as many first bypass lines 28 as there are conduits 4. Starting from its opening 282, each first bypass line 28 is equipped with a filter 30 and with a stop valve 32. The first four lines 28 join up downstream from their respective stop valves 32 and the tapping connection 34 is common to the first four bypass lines 28, as well as their opening 284 into the buffer reservoir 26.

A tapping connection 8 is provided on each conduit 4 and equipped with a valve 10 manually controlled, according to an approach parallel to the one mentioned above concerning the first embodiment. Alternatively, only one or certain conduits 4 and/or are equipped with such a tapping connection 8.

The second discharge line 42 is common for all the cylinders of the piece of equipment, in particular of the engine, and receives, downstream from the reservoir 26, the oil from all the first bypass lines 28. The opening 424 of the second discharge line is positioned on one of the conduits 4, downstream from its stop valve 20.

This third embodiment gives the possibility of optimizing the congestion of the conduits 4 and their path within the engine compartment of a ship. It allows a gain in room relatively to the first embodiment.

By successively applying the explicit method above concerning the first embodiment, for each of the conduits 4, the installation of this third embodiment gives the possibility of determining, by means of the sensor 50, identical with the one of the first embodiment, the overall iron content of the lubricant at the outlet of each of the cylinders of the piece of equipment M onto which is connected a conduit 4.

In the example of FIG. 19, four conduits 4 are provided, each dedicated to a cylinder of the piece of equipment M. Alternatively, the number of conduits 4 is different, while remaining greater than or equal to 2, in order to adapt the installation 2 depending on the configuration of the piece of equipment M and on the room available for housing the conduits 4.

Alternatively, the invention may be illustrated with the sensors 46, 48 and 50 positioned in this order along the discharge line 42. This is not mandatory and another order may be contemplated. For example, the sensor 50 may be positioned upstream from the sensors 46 and 48, or between the latter, along the line 42.

Regardless of the embodiment, the sensors 46 and 48 are optional, in the sense that they are not useful for determining the overall iron content of the oil. They are however very advantageous since they allow the installation 2 to automatically carry out several measurements of several representative parameters of the quality of the oil, in addition to the single content of dissolved and/or particulate iron. Thus, the installation 2 allows an efficient measurement of the overall iron content, of the basicity index or BN and/or of other parameters of an oil leaving the piece of equipment M by means of a method which may be automated and which does not require any particular knowledge on behalf of a user or seagoing personnel, since the signal S2 may be directly legible, either by a person or by a machine.

In practice, the maximum pressure P1′ prevailing in the inner volume V26 of the reservoir 26, which depends on the pressure of the source 12, is not limited to 7 bars. It is comprised between 6 and 12 bars, preferably between 7 and 10 bars depending on the pressure of the compressed air network available on the ship. The value of 7 bars is preferred since it gives good experimental results and corresponds to a currently available pressure level. It is important that this pressure P1 be greater than the pressure P4 of the oil in the conduit 4, which is comprised between 1.1 and 6 bars as mentioned above. Indeed, it is the difference between the pressures P1 and P4 which ensures the flow of the oil through the second discharge conduit 42.

Regardless of the embodiment, the installation 2, which is essentially comprised in the box 24, is easy to install onboard a ship and does not require the setting into place of the valve 20 in the conduit 4, the connection of the lines 28 and 42 on this conduit and its supply with current and pressurized air. The installation 2 may therefore be easily implanted on a new ship or used for retrofitting a ship in operation.

The invention is described above in the case when the sensor 50 is used for determining the overall content of iron of the lubricant. It is however applicable for determining the overall content of a lubricant in another predetermined chemical element, for example in calcium, vanadium, chromium, molybdenum, sulfur, lead, copper, silver, tin, aluminium, nickel, zinc or phosphorus. In this case, the constitutive material of the casing is adapted to the relevant chemical element, so as not to perturb the X fluorescence measurement.

The invention is described above in the case of its use for an engine for propelling a ship. It is however applicable to other ship equipment, for example an auxiliary motor or an accessory of a ship.

In the foregoing, the words and expressions «oil», «engine oil» and «lubricant» are used indistinctly since, in the sense of the invention, an oil or an engine oil is a lubricant.

In the foregoing, the expressions «iron content», «overall iron content» and «content of dissolved and/or particulate iron» are used indistinctly, as well as the expressions «content in a predetermined chemical element» and «overall content in a predetermined chemical element».

The features of the embodiments and contemplated alternatives above may be combined for generating new embodiments of the invention.

1. An installation for following up the evolution of the quality of a lubricant contained in a piece of equipment, this installation comprising:
at least one conduit for circulating the lubricant, this conduit being connected, upstream, to the piece of equipment and downstream, to a recovery pan, and
a sensor for determining, by the X fluorescence technique, the overall content in a predetermined chemical element, of a lubricant sample, this sensor comprising an X-ray source, an X-ray detector and a cell intended to contain a lubricant sample to be analyzed and equipped with a wall forming a window for letting through the rays stemming from the source or going towards the detector;
characterized wherein
in that the installation comprises:
a first controlled interruption valve for the circulation of the lubricant in the conduit,
a buffer reservoir for accumulating lubricant,
a first bypass line, connected on the one hand to the conduit, upstream from the first valve and, on the other hand to the buffer reservoir,
a second controlled interruption valve for the circulation of the lubricant in the first bypass line,
a second discharge line for the lubricant, from the buffer reservoir to the recovery pan,
a third controlled interruption valve for the circulation of the lubricant in the second discharge line
in that the sensor determines the overall content in a predetermined chemical element, of a lubricant sample, at the outlet of the buffer reservoir; and
in that the wall, which forms a window for letting through the X-rays from the source or going towards the detector, is made in poly.

2. The installation according to claim 1, wherein the cell comprises a metal casing on which the wall is added.

3. The installation according to claim 2, wherein the cell comprises a hollow housing for receiving the wall and a threaded washer for immobilization of the wall in the hollow housing.

4. The installation according to claim 1, wherein the sensor is a sensor for determining the iron content of the lubricant sample.

5. The installation according to claim 2, wherein the casing is made in a metal or in a non-ferrous alloy.

6. The installation according to claim 2, wherein the X-ray source and the X-ray detector are mounted on a lid which positions them relatively to the housing and to the wall and wherein this support forms a shield against the propagation of X-rays.

7. The installation according to claim 1, wherein a targeting axis of the X-ray source and a targeting axis of the X-ray detector form between them an angle comprised between 20 and 25°.

8. The installation according to claim 1, wherein the wall has a thickness of less than or equal to 200 μm.

9. The installation according to claim 1, wherein the sensor for determining the content in a predetermined chemical element is positioned on the second discharge line.

10. The installation according to claim 1, wherein it further comprises a sensor for determining the basicity index of the lubricant, positioned on the second discharge line and which allows determination of the basicity index of the lubricant at the outlet of the buffer reservoir.

11. An automated method for following up the evolution of the content in a predetermined chemical element of a lubricant circulating in a piece of equipment, by means of an installation according to claim 9, said method comprising at least steps consisting of:
a) closing the first valve,
b) opening the second valve and closing the third valve for supplying the buffer reservoir from an amount of lubricant accumulated in the conduit, upstream from the first valve,
c) opening the third valve for having the lubricant present in the buffer reservoir circulate through the second discharge line as far as into a cell of the sensor,
d) using the sensor for determining the overall content in a predetermined chemical element of the lubricant at the outlet of the buffer reservoir.

12. A method for following up the operation of a piece of equipment loaded onboard a ship, comprising the determination, onboard the ship, of the overall content in a predetermined chemical element of a lubricant circulating in the piece of equipment by applying a method according to claim 11.

13. The method according to claim 12, wherein the method is performed to determine an overall content in a predetermined chemical element of a lubricant circulating in a piece of equipment of a ship.

14. The installation according to claim 7, wherein the angle between the targeting axes of the X-ray source and the X-ray detector is of the order of 22°.

15. The installation according to claim 8, wherein the wall has a thickness less than or equal to 150 μm.

16. The installation according to claim 8, wherein the wall has a thickness of the order of 125 μm.

17. The installation of claim 5, wherein the casing is made in an alloy based on aluminum.

18. The method of claim 12, wherein the predetermined chemical element is iron.

19. The method of claim 13, wherein the predetermined chemical element is iron.

20. The method of claim 19, wherein the piece of equipment is a ship engine.
